(19) United States
(12) Patent Application Publication
(10) Pub. No.: US 2006/0247479 A1
(43) Pub. Date: Nov. 2, 2006

(54) Ethyl benzene from refinery grade feedstocks

(75) Inventors: Rajnikant R. Barchha, Moon Township, PA (US); Ronald Scott Smith, Calgary (CA)

Correspondence Address: Suzanne Kikel;NOVA Chemicals Inc., 400 Frankfort Road, Monaca, PA, 15061, US

(73) Assignee: Nova Chemicals inc.

(21) Appl. No.: 11/119,185

(22) Filed: Apr. 29, 2005

(51) Int. Cl. C07C 2/68

(52) U.S. Cl. 585/467

(57) ABSTRACT

“Chemical grade” ethyl benzene is synthesized by the alkylation of the benzene which is contained in the reformate stream of a refinery with the ethylene in FCC (fluid catalytic cracker) off gas. The reformate stream is not hydrogenated prior to the alkylation. The alkylation reaction takes place in a fixed bed of particulate catalyst. The catalyst is preferably a zeolite, especially zeolite beta. The preferred reactor is a catalytic distillation reactor. The process of this invention allows (but does not require) the reformer to be operated under severe conditions (which produces high octane gasoline components but which also lead to high benzene concentrations in the reformate), yet still meet environmental regulations on gasoline (because the process removes substantially all of the benzene from the gasoline). The ethyl benzene is removed from the reformate stream and may be used for the production of styrene.

### FIELD OF THE INVENTION

This invention relates to the preparation of ethylbenzene using dilute, refinery-grade feedstocks.

### BACKGROUND OF THE INVENTION

Ethylbenzene is a commodity chemical of significant commercial importance. It is typically prepared by the alkylation of “chemical grade” (or “high purity”) benzene with chemical “grade” ethylene and its primary use is to produce styrene.

The use of less pure feedstocks (i.e. “dilute benzene” and/or “dilute ethylene”) has been proposed as a way to reduce the cost of producing ethylbenzene.

Another approach to reduce ethylbenzene production costs is to integrate the operation of an ethylbenzene plant with an ethylene cracker and/or a refinery in order to make more efficient use of certain unit operations (such as hydrogenation, extraction and fractionation) in the integrated process than would be possible if the two were operated separately.

Finally, from a refiners' perspective, there is a desire to separate olefins and benzene from the gasoline pool in order to satisfy regulatory mandates. That is, gasoline which has been treated so as to remove olefins and/or benzene is more “environmentally friendly” than the untreated gasoline.

The alkylation of benzene contained in the gasoline pool can be a cost effective way to meet this objective. In particular, the alkylation product is more readily fractionated out of the gasoline in comparison to benzene.

Thus, in summary, it has been previously proposed to produce ethylbenzene using dilute feedstocks with “integrated plant operations” (and as a means to improve the quality of gasoline). For example:

U.S. Pat. No. 4,975,179 (Harandi et al.; assigned to Mobil Oil Corporation) discloses the alkylation of the benzene contained in the reformate stream from a catalytic cracker using a zeolite catalyst. This reduces the benzene content of the gasoline. The resulting alkylate may be left in the gasoline to enhance the octane rating of the gasoline.

Similarly, U.S. Pat. No. 5,002,990 (Hsieh et al.; assigned to Chevron Research and Technology) teaches a company process for reducing the content of benzene in a reformate stream by alkylating the reformate with an olefin stream. The process claimed by Hsieh et al. '990 employs a zeolite catalyst and is conducted in a catalytic distillation reaction. The disclosure of this patent also refers to an earlier paper entitled “Alkylation of FCC Off-Gas Olefins with Aromatics via Catalytic Distillation” (the applicant could not obtain a copy of this reference).

U.S. Pat. No. 6,002,058 (Hearn et al.; assigned to Catalytic Distillation Technologies) teaches the alkylation of refinery-grade benzene with lower olefins. The benzene-containing stream is first hydrogenated in order to remove impurities, especially sulfur compounds.

U.S. Pat. No. 5,750,814 (Grootjans et al.; assigned to Fina Research S.A.) teaches the alkylation of aromatic compounds (which are preferably obtained from a refinery) with an alkylation agent. The process of Grootjans et al. is similar to the process of Hearn et al. in that the aromatic feedstock is selectively hydrogenated prior to being fed to the alkylation reactor.

Likewise, U.S. Pat. No. 6,002,057 (Hendricksen et al.; assigned to Exxon Chemical Patents Inc.) teaches a process for the alkylation of (preferably a refinery-grade) aromatic stream with an olefin stream using a specific zeolite catalyst, namely zeolite beta. The process of Hendricksen et al. also expressly requires the hydrogenation of the aromatic stream.

Thus, the prior art does disclose various processes for the alkylation of refinery-grade aromatics. As noted above, the processes of Hearn et al., Grootjans et al., and Hendricksen et al. specify that the aromatic stream must be hydrogenated prior to being introduced into the alkylation reactor.

### SUMMARY OF THE INVENTION

The present invention provides a process to prepare ethyl benzene from refinery grade benzene comprising the reaction of:

(a) a non-hydrogenated reformate stream containing from 20 to 80 weight % benzene; and

(b) a dilute ethylene stream comprising from 50 to 80 mole % ethylene, in an alkylation reaction, in the presence of a particulate alkylation catalyst, under alkylation conditions whereby mono and poly ethyl benzenes are formed; and

(c) separating said mono and poly ethyl benzenes from other unreacted hydrocarbons.

### DETAILED DESCRIPTION

A. Dilute Benzene

The process of this invention uses a dilute benzene stream from a refinery source. In general, dilute benzene may be available at a refinery in (i) a coker gasoline; (ii) a catalytic cracker naphtha stream; or (iii) a reformate stream. However, the process of this invention is specifically limited to the use of “reformate” as the dilute benzene source. Most preferably, the reformate which is used in the process of the present invention is obtained from a reformer which operates with a precious metal catalyst (especially a platinum/rhenium catalyst). It is preferred that the feed to the reformer is pre-treated in a manner which serves to protect the catalyst (e.g. a hydrotreating step). The resulting catalytic reformate stream will generally have a density of from about 0.7 to 0.9 grams per cubic centimeter, a boiling range between about 150° C. to about 205° C., a C₈ aromatic content between about 4 and about 60 mole %, a toluene content of about 2 to about 60 mole %, a benzene content of about 1 to about 60 mole % and (in addition) paraffins, and other aromatics.

The dilute benzene stream which is used in the process of this invention must contain from about 20 to about 80 weight % benzene, preferably from 20 to 70 weight % benzene. This requirement may necessitate that the catalytic reformate is fractionated to a lighter, narrow cut reformate comprising mainly of C6 hydrocarbons so as to increase the benzene concentration before it is introduced into the alkylation unit.

However, it is not necessary to hydrogenate the catalytic reformate in order to prepare the dilute benzene stream. This eliminates a unit operation (i.e. hydrogenation) from prior art processes to alkylate dilute benzene. In addition, this improves the hydrogen balance within the overall refinery and leaves hydrogen available for other hydrogenation operations in the refinery.

B. Dilute Ethylene

The process of this invention uses a dilute ethylene stream to alkylate the above described dilute benzene stream. The dilute ethylene stream which is used in the alkylation reaction preferably containing from 50 to 80 mole % ethylene (most preferably, from 60 to 75 mole % ethylene). Non-interfering diluent gases, such as methane, C₂ to C₄ paraffins, hydrogen and carbon oxides (i.e. gases which do not have a substantial adverse impact on the akylation reaction) may also be present. A preferred dilute ethylene stream comprises at least 95-99 mole % (ethylene plus ethane) (with the requirement that the ethylene concentration is from 50 to 80 mole %) and less than 5 mole % other non-interfering diluent gases.

In a preferred embodiment, the dilute ethylene stream is the product of a fluid catalyst cracking (“FCC”) but other sources would also be suitable, including the product of thermal cracking of ethane or hydrocarbon liquid feedstocks (e.g. naphtha).

C. Alkylation Reaction

The process of this invention requires the alkylation of the dilute benzene stream defined above with the defined dilute ethylene in the presence of a particulate catalyst.

Preferred alkylation (and transalkylation) catalysts are zeolites selected from the group consisting of ZSM-4, zeolite omega, zeolite beta, zeolite γ and modifications thereof. Zeolite beta having a high surface area and low sodium content is preferred.

All of the above noted zeolites are well known to those skilled in the art and are extensively described in the patent literature (U.S. Pat. Nos. 4,975,179; 5,002,990; 6,002,057; 6,002,058; and 5,750,814), the disclosures of which are incorporated herein by reference.

Various types of reactors are known for alkylation reactions. For example: alkylations may take place in a fixed bed (or moving bed); batchwise or continuously; in an up-flow (or down-flow) arrangement with co-current (or countercurrent reaction flow). In addition, it is known to use multi-stage addition of olefin.

The process of this invention preferably is conducted in a fixed bed reactor. Most preferably, the reactor takes place in a so-called “catalytic distillation reactor” (examples of which are disclosed in U.S. Pat. Nos. 5,082,990; 6,002,057; and 6,002,058).

The process of this invention further requires that ethyl benzene is separated from the other by-products of the alkylation reaction. In this manner, benzene is removed from the gasoline pool and an ethylbenzene stream is available for chemical production.

The “recovery” of the chemical grade ethylbenzene is further described below.

D. “C2” Recovery

The alkylation product contains a large volume of light diluents (primarily, the ethane from the dilute ethylene). These lights are removed from the alkylation product by distillation by a process of deethanization, a process which is well known to those versed in the art.

E. Ethyl Benzene Recovery

Preferred process for the recovery/purification of ethyl benzene are described below. The crude alkylation product is subjected to a distillation process that separates the crude alkylation product into:

1) a monoethylbenzene stream;

2) a polyethylbenzene stream; and

3) a heavy residue.

(For clarity: when operating a catalytic distillation reactor, the crude alkylation product may be recovered as a bottoms stream from the reactor. This bottoms stream is then separated into the three streams noted above.)

In a preferred embodiment, the polyethylbenzene stream is then reacted with benzene under transalkylation conditions to produce a second monoethylbenzene stream. The monoethylbenzene streams are preferably combined and then sent to a styrene production facility.

F. Aromatics Separation

The remainder of the crude alkylation product still contains unreacted benzene in addition to toluene and varying amounts of C2 to C7 paraffins and cycloparaffins. This stream may be used as a benzene reduced reformate suitable for gasoline. Alternately, this stream may be further processed to separate benzene from the other components for recycle to the alkylation process. Preferred benzene separation techniques include distillation, extractive distillation and solvent extraction, all of which are well known to those of ordinary skill in the art.

The process of the invention will now be illustrated by the following non-limiting examples.

### EXAMPLES

Features of the invention are further illustrated by the following non-limiting examples.

### Example 1—Comparative

### Pure Component Alkylation

A 450 mL autoclave reactor was charged with 0.75 g of activated beta zeolite and 106 mL of pure benzene. The reactor was sealed and purged with nitrogen. The reactor was pressurized with ethylene to 145 psig. The reactor was heated to 215° C. and held at that temperature for 6 hours. The liquid product of the batch reaction was analyzed and found to contain 67.1% by weight benzene, 28.4% ethylbenzene, and 4.4% C10 and heavier species, indicating a 27.5% benzene conversion with selectivity to ethylbenzene of 89%.

### Example 2

### Preparation of Model Reformate

A “model” (or “pseudo”) reformate base was prepared having the following composition:

| Component | Weight % |
|---|---|
| Iso-pentane | 0.6 |
| Normal-pentane | 1.9 |
| Iso-hexane | 28.6 |
| Normal-hexane | 35.5 |
| Cyclopentane | 4.5 |
| Cyclohexane | 12.5 |
| Methylcyclopentane | 10.5 |
| Normal-heptane | 3.4 |
| Toluene | 2.6 |

Model reformates having 20% and 50% benzene by volume were prepared by adding 1 part by volume benzene to 4 parts model reformate base and 1 part benzene by volume to 1 part model reformate base, respectively.

### Example 3—Inventive

### Batch Alkylation of 50% Benzene Reformate with 75% Ethylene

A 450 mL autoclave reactor was charged with 0.75 g of activated beta zeolite and 147 mL of 50 volume % benzene model reformate. The reactor was sealed and purged with nitrogen. The reactor was pressurized with a 75 mole % ethylene/25% ethane mixture to 103 psig to provide a benzene to ethylene mole ratio of approximately 3.5. The reactor was heated to 215° C. and held at that temperature for 6 hours. Analysis of the liquid product of the batch reaction indicated a 25.3% benzene conversion with selectivity to ethylbenzene of 87.9%. A toluene conversion of 19.6% was observed.

### Example 4—Inventive

### Batch Alkylation of 20% Benzene Reformate with 60% Ethylene

A 450 mL autoclave reactor was charged with 0.75 g of activated beta zeolite and 291 mL of 20 volume % benzene model reformate. The reactor was sealed and purged with nitrogen. The reactor was pressurized with a 60 mole % ethylene/40 % ethane mixture to 64 psig to provide a benzene to ethylene mole ratio of approximately 3.5. The reactor was heated to 215° C. and held at that temperature for 6 hours. Analysis of the liquid product of the batch reaction indicated a 12.2% benzene conversion with selectivity to ethylbenzene of 89.0%. A toluene conversion of 22.7% was observed.

### Example 5—Inventive

### Batch Alkylation of 20% Benzene Reformate with 60% Ethylene at 235° C.

A 450 mL autoclave reactor was charged with 0.75 g of activated beta zeolite and 108 mL of 20 volume % benzene model reformate. The reactor was sealed and purged with nitrogen. The reactor was pressurized with a 60 mole % ethylene/40 % ethane mixture to 45 psig to provide a benzene to ethylene mole ratio of approximately 3.5. The reactor was heated to 235° C. and held at that temperature for 6 hours. Analysis of the liquid product of the batch reaction indicated a 15.9% benzene conversion with selectivity to ethylbenzene of 84.0%. A toluene conversion of 26.7% was observed.

What is claimed is:

1. A process to prepare ethyl benzene from refinery grade benzene comprising the reaction of:
(a) a non-hydrogenated reformate stream containing from 20 to 80 weight % benzene; and
(b) a dilute ethylene stream comprising from 50 to 80 mole % ethylene, in an alkylation reaction, in the presence of a particulate alkylation catalyst, under alkylation conditions whereby mono and poly ethyl benzenes are formed; and
(c) separating said mono and poly ethyl benzenes other unreacted hydrocarbons.

2. The process according to claim 1 wherein said process further comprises transalkylating said poly ethyl benzenes with benzene.

3. The process according to claim 1 wherein said process is in a fixed bed alkylation reactor.

4. The process according to claim 1 wherein said catalyst is a zeolite.

5. The process according to claim 4 wherein said zeolite is zeolite beta.

6. The process according to claim 1 wherein said alkylation conditions are conducted in a catalytic distillation reactor.

7. The process according to claim 6 wherein said mono and poly ethyl benzenes are separated into:
(i) a monoethylbenzene stream;
(ii) a polyethylbenzenes stream; and
(iii) a heavy residue;
and wherein said polyethylbenzenes stream is reacted with benzene under transalkylation conditions so as to produce a second monoethylbenzene stream.

8. The process according to claim 1 wherein said mono and poly ethyl benzenes are separated into:
(i) a monoethylbenzene stream;
(ii) a polyethylbenzenes stream; and
(iii) a heavy residue;
and wherein said polyethylbenzenes stream is reacted with benzene under transalkylation conditions so as to produce a second monoethylbenzene stream.

9. The process according to claim 1 wherein a stream containing unreacted benzene is separated from said mono and poly ethyl benzenes and recycled to said alkylation reactor.

10. The process of claim 1 wherein said dilute ethylene stream consists of from 60 to 75 mole % ethylene, less than 5 mole % of non-interfering diluent gases and the balance ethane.
